(19) United States
(12) Patent Application Publication
(10) Pub. No.: US 2025/0387326 A1
(43) Pub. Date: Dec. 25, 2025

(54) CRYSTALLINE PHARMACEUTICAL COMPOSITION FOR INHALATION COMPRISING SUGAR AND LIPID COMPOSITE PARTICLES AND PROCESS FOR MANUFACTURE

(71) Applicant: HOVIONE SCIENTIA LIMITED, Cork (IE)

(72) Inventors: Beatriz FERNANDES, Lisboa (PT); Rodrigo AMORIM, Amadora (PT)

(21) Appl. No.: 18/877,710

(22) PCT Filed: Jun. 21, 2023
(86) PCT No.: PCT/GB2023/051623

§ 371 Date: Dec. 20, 2024

(30) Foreign Application Priority Data
Jun. 21, 2022 (PT) 118060

(51) Int. Cl. A61K 9/00; A61K 9/14; A61K 31/4196; A61K 31/706; A61K 47/26; A61K 47/28

(52) U.S. Cl. CPC A61K 9/0075; A61K 9/145; A61K 31/4196; A61K 31/706; A61K 47/26; A61K 47/28

(57) ABSTRACT

The present invention describes a pharmaceutical composition comprising composite particles with a controlled aerodynamic particle size distribution, wherein the composite particles comprise one or more active pharmaceutical ingredients (API), at least one sugar and at least one lipid. A process for manufacturing the pharmaceutical composition comprises the steps of: a. Blending API and one or more excipients comprising at least one sugar or at least one lipid, or both at least one sugar and at least one lipid, into a homogeneous powder; b. Reducing the particle size distribution of the blend. The micronized pharmaceutical composition allows for the delivery of crystalline stable API with better aerodynamic properties than the micronized API alone, as well as improved downstream processing and stability properties.

### TECHNICAL FIELD OF THE INVENTION

The present invention relates in general to the field of pharmaceutical dry powders, and more particularly to pharmaceutical compositions of particles, in particular composite particles, which have enhanced aerodynamic performance for inhalation delivery.

One of the major advantages of pulmonary drug delivery is the rapid clinical onset, due to the high surface area of the lung (>100 m2) coupled with high irrigation with blood (with a flowrate as high as 5.7 L/min) and a thin absorption membrane (0.1-0.2 μm) [1]. Moreover, delivering drugs to the lung enables first-pass metabolism by-passing, increasing bioavailability and reducing the required dose, which in turn decreases the therapeutic cost. Pulmonary drug administration has been used for years for low dosage delivery to treat conditions like asthma. High dosage delivery by inhalation is interesting in that many new drugs present low bioavailability when administered orally due to low solubility or absorption. In addition, for local delivery, pulmonary delivery presents a decrease in systemic side effects and a higher concentration at the site of action, and regarding systemic delivery, it improves the administration of labile molecules in a non-invasive way.

Pressurized metered-dose inhalers, nebulizers and dry powder inhalers (DPIs) are the usual devices to deliver drugs to the lung. Among these, the latter have the benefits of being propellant free, not requiring coordination of actuation with inhalation, being portable and relatively inexpensive, and keeping the drug in the solid state, which presumably provides higher physical-chemical stability [2].

Inhalation powders are required to be within a specific particle size distribution (PSD) (50% of particles in volume (Dv50) should be below 5 μm) and have a median mass aerodynamic diameter (MMAD) of 1 to 5 μm so the deep airways are targeted. Fully crystalline and moisture-free powders are preferred for increased stability.

The performance of DPI formulations is usually assessed through the emitted dose (ED), which is the mass of powder per capsule (mg/caps) that leaves the capsule upon actuation, and the fine particle dose (FPD), which is the mass of powder per capsule (mg/caps) that flows through a cutoff aerodynamic diameter of 5 μm upon actuation. The fine particle fraction (FPF) is another performance indicator, obtained by dividing the FPD by either the ED or the label mass claim. All the measurements of these performance indicators should reveal a relative standard deviation (RSD) below 5%.

Powder flow properties are dependent on particle size distribution, bringing the major drawback of the pulmonary administration route, which requires reduced particle sizes to target the deeper airways. The performance of a fine powder is affected by particle size in that the relative importance of interparticulate forces to gravitation forces increases when particle diameter decreases: whilst gravitational forces are proportional to the cube of particle size, van der Waals forces are directly proportional to the particle size, so upon size reduction, the latter gain relevance. Besides, electrostatic forces, capillary forces and mechanical interlock, are other relevant interparticle interactions, also dependent on particle size and shape, as well as on surface texture and contact area, surface energy, hygroscopicity and relative humidity [2]. Particle shape can limit the approach of two particles, thereby reducing interparticle interactions. The same effect can occur due to surface roughness: in fact, surface asperities in the order of 1 μm limit the van der Waals to negligible values [3]; when the particles are planar or elongated, the opposite happens, as intimate contact is allowed. In case the size of the asperities is large enough such that entrapment of other particles can occur, mechanical interlocking and uneven distribution of surface energy occur. Relative humidity plays a role in two opposing mechanisms as it increases interactions due to capillary forces (which can be as strong as the more hygroscopic the material is), but also increases conductivity, thereby dissipating electrostatic charges. Electrical charge arises from collision and friction amongst the particles during powder mixing and other handling processes [2]. In general, van der Waals forces are predominant, and all these interactions are only relevant for particles with diameters in the order of a few micrometers or smaller, which are thereby more prone to become cohesive and agglomerate.

When delivering low doses, the most common strategy to ensure acceptable aerosolization and deal with the inherent cohesion of fine powders is to use a coarser inert carrier such as lactose that dissociates after inhalation, remaining in the device or depositing in the mouth/upper airways, enabling the drug particles to re-disperse in the airflow. For high dosage formulations (typically <5 mg of delivered API), the use of a carrier is not suitable as active site saturation of the carrier causes undesirable particle segregation. An alternative approach is to produce carrier-free soft aggregates of API, which remain intact through the handling process but are easily de-agglomerated upon inhalation. However, the inherent cohesiveness of fine powders brings a wide variability to the carrier-free approach, as the formation of stable agglomerates may occur, which do not de-agglomerate upon actuation, thereby not reaching the lower airways, and significantly reducing the actual delivered dose [4].

Co-milling stands for the co-processing of two or more types of particles (e.g. an API and a lubricant) for the production of composite particles with enhanced performance. The benefits obtained are often due to the dispersion of additive particles on the surface of API particles. In addition, co-milling can be used to enhance the absorption of poorly soluble drugs such as previously referred itraconazole, namely by providing composite particles with increased wettability [5]. The excipients used in co-milling comprise a number of different types of compounds, which will act through different mechanisms [10][9].

The improvements observed in the aerodynamic performance of co-milled formulations have been explained by the adhesion of excipient particles to the high surface energy sites of API particles [6], acting as spacers, reducing contact area and hindering interparticle interactions. In some cases, the excipient particles might orient hydrophobic groups towards the exterior, sometimes forming a coating film [7]. In general, studies explain the improvement in the FPF by the reduction in surface energy [8][9]. Electrostatic stabilization has also been reported as a mechanism of preventing particle agglomeration through repulsion between particles, providing long term particle size stability [10].

In regard to the prevention of solid-state transformations and crystal defects that often occur during milling, co-milling has shown promising results [11][12]. Mechanically induced amorphization competes with thermodynamically induced re-crystallization, thereby, in a general way, milling below glass transition temperature of a material most likely results in an amorphous product as it provides conditions for the amorphous state to be preserved after it is induced by the disordering effect of shearing. On the other hand, using co-milling techniques to reduce the overall glass transition temperature of a pharmaceutical composition the efficiency of re-crystallization is of an order of magnitude that no amorphization is observed. Crystalline formulations are desirable in that the amorphous state is inherently unstable, leading to re-crystallization, which can promote the formation of solid bridges and consequent agglomeration. In addition, the amorphous state is more prone to cause water sorption, requiring more strict storing conditions. Moreover, controlling the crystalline state of the API can lead to a controlled release by avoiding the super saturation of the amorphous form in the pulmonary lining fluid, and thus available for therapeutic effect. This presents one of the major benefits of milling processes when compared to spray-drying, which is known to yield amorphous products.

Stability is a critical attribute of pharmaceutical powders. An increase in the particle size due to cohesive forces, water sorption due to amorphous regions, subsequent solid bridge formation or drug degradation are all undesirable effects. Co-milling is potentially a promising approach in increasing drug product stability in that it can be applied to seek to minimize these effects, as aforementioned. Also, it is possible for some excipients to form a hydrophobic coating film that protects from humidity and from degradation [7].

Because smaller particles have larger surface area, thereby being dissolved more quickly, particle size reduction alone becomes a promising method for dissolution enhancement. The use of wetting agents in co-milling may present itself as an improved method for this purpose by the incorporation of a substance that reduces the surface tension of water, allowing it to spread onto the surface more readily. When these particles are readily dissolved, it is possible that the composite particle becomes a porous API particle, increasing the contact area even further.

U.S. Pat. No. 8,802,149B2 relates to pharmaceutical compositions comprising active ingredient, a hydrophilic and a hydrophobic compound, for inhalation, produced by spray drying. Spray-dried formulations comprising hydrophilic and hydrophobic materials have been studied in the literature [15][16]. However, contrarily to, for example, jet-milling, this method is known to yield completely amorphous products, which are more prone to water sorption and stability issues that can be critical in inhalation formulations, where the particle/agglomerate size determines the delivered dose. Besides, spray-drying is a more complex process when compared to most milling processes, involving the optimization of several steps (dissolution, atomization and collection) and the use of solvents.

U.S. Pat. No. 8,182,838B2 describes the method of jet milling active particles in the presence of particles of an aminoacid, a metal stearate and/or a phospholipid to form composite active particles, further comprising blending carrier particles with the composite active particles. The carrier-based approach, however, is not suitable for high dosages, as aforementioned. Besides, aminoacids' safety for pulmonary delivery is not recognized, and the hydrophobicity of metal stearates and phospholipids can be detrimental to dissolution, their prolonged residence time in the airways causing irritation, in particular for metal stearates. U.S. Pat. No. 8,932,635B2 depicts the surface coating of active particles for inhalation delivery with magnesium stearate with the intent of delaying dissolution.

EP1663155B1 describes the co-jet-milling method to produce composite particles for pulmonary delivery, the excipients comprising an aminoacid, a metal stearate or a phospholipid which coat the active particles. These materials carry the aforementioned drawbacks.

U.S. Ser. No. 11/103,448B2 describes the method of milling particles of a metal stearate and particles of active material separately, and jet-milling both previously milled active and metal stearate particles to yield composite particles for inhalation. This method carries the disadvantage of comprising several steps and the aforementioned hydrophobicity of metal stearates and airway irritation.

Lo et al. produced carrier based particles for inhalation with enhanced performance by spray-drying liposomes of API particles together with sugars (sucrose, trehalose, and lactose), with stabilizing function, and lipids (DMPC, DPPC, DSPC, or DPPG) [13]. As aforementioned, the carrier-based approach is not suitable for high dosages. Besides, this process comprises several steps.

US2007178166 describes methods for making a dry powder pharmaceutical formulation for pulmonary or nasal administration. Particles of API are blended with one first excipient to form a first powder blend, which is then milled, and subsequently in a second step the milled blend is blended with a second excipient to form a blended dry powder. The particles of the second excipient are larger than the microparticles or nanoparticles in the milled blend.

WO2022126105A1 discloses a method, composition, and kit for the treatment of fibrotic lung disease. The method utilizes a combination product for inhalation comprising a dry powder formulation provided in an inhaler to be administered by oral inhalation. The composition comprises diketopiperazine particles, and the pharmaceutical dry powder is prepared by spray-drying.

CN106102748A discloses dry powder formulations comprising acetylsalicylic acid particles and includes milling and spray-drying steps.

US2006257491A1 describes mechanofusion and jet-milling for the production of dry powder for pulmonary inhalation. Blends comprise API and additive material such as aminoacid/metal stearate/phospholipid. The formulations described include leucine (aminoacid) or magnesium stearate (metal stearates) which may present safety issues for pulmonary delivery or irritation due to the hydrophobicity of compounds, respectively.

KR20190068591 describes dry particles comprising a crystalline particulate antifungal agent and focusses on the preparation of a crystalline drug treated with an anti-solvent and a stabilizer to form a suspension. There is no disclosure of milling blends of different components to improve aerodynamic performance and/or stability.

### DESCRIPTION OF THE INVENTION

In a broad aspect, the present invention provides a pharmaceutical composition comprising one or more active pharmaceutical ingredients (API), at least one sugar and at least one lipid. The composition has a controlled aerodynamic particle size distribution, owing to the method of manufacture. The API is in crystalline form. Suitably, the other components of the composition may also be in crystalline form. For example, either or both of the sugar and lipid component may be in crystalline form.

Suitably, the composition comprises composite particles. Such particles are composed of the active ingredient and at least two excipients in individual particles. Preferred particles are composite particles comprising API, a sugar component and a lipid component. The composition is preferably made by co-milling.

In a further aspect, the invention thus provides a pharmaceutical composition comprising composite particles, wherein the composite particles comprise one or more active pharmaceutical ingredients (API) in crystalline form, at least one sugar and at least one lipid. The particles have a controlled aerodynamic particle size distribution. Composite particles prepared by co-milling, wherein the composite particles comprise one or more active pharmaceutical ingredients (API), at least one sugar and at least one lipid, are thus an aspect of the invention. Thus, co-milled composite particles comprising one or more active pharmaceutical ingredients (API), at least one sugar and at least one lipid are provided. The invention thus provides, in one aspect, crystalline composite particles.

Suitably, co-milling is used to obtain the particles of the composition. Co-milling is for example reported as the co-processing of API/excipient with the additive material for the production of composite particles (for example, reference can be made to [18] Lau et al, 2017). Co-milled particles as described are thus an aspect of the invention. One aspect of the present invention is thus co-milling of API, sugar (such as for example mannitol) and lipid (such as for example cholesterol) together, to provide composite particles. Such particles are preferably crystalline.

The invention also provides a pharmaceutical composition as disclosed and claimed herein for use as a medicament. For example, the pharmaceutical composition may be for use in the treatment of a pulmonary condition in a patient.

The presently disclosed composition may, for example, be used in a dry powder inhaler, as will be understood by those skilled in the art. Any suitable dry powder inhaler may be used.

Accordingly, the invention also provides a dry powder inhaler comprising a pharmaceutical composition as disclosed and claimed herein.

In a further aspect, there is also provided a process for manufacturing a pharmaceutical composition as disclosed and claimed herein, which process comprises the steps of:
a. Blending API and one or more excipients comprising at least one sugar or at least one lipid, or both at least one sugar and at least one lipid, into a homogeneous powder;
b. Reducing the particle size distribution of the blend.

In a preferred aspect, step (b) is carried out without the use of a solvent.

Step (b) preferably comprises co-milling of the particles. It also preferably comprises jet-milling, although other similar methods may be used if desired. For example, co-miling by a wet milling method may be used. High pressure homogenisation can for example be a useful method in the context of this invention, as explained below.

The invention also provides composite particles with a controlled aerodynamic particle size distribution when prepared by the method of the invention, The composite particles comprise one or more active pharmaceutical ingredients (API), at least one sugar and at least one lipid. A pharmaceutical composition comprising such composite particles is also provided. Suitably, the components of the composition are crystalline.

The present invention thus relates to a pharmaceutical composition of composite particles comprising at least one API with at least one sugar and at least one lipid produced by co-milling for use in inhalation formulations with improved performance by hindering interparticle interactions and preventing cohesion. We have found that the performance enhancement obtained by using said pharmaceutical composition is reflected in better stability, reduced amorphization and improved dissolution, all with minimal amounts of additive material. Sugars have been widely employed in DPI as carriers and are known for improving wettability. However, surprisingly, we have found that sugars are able to improve the FPF of co-milled formulations, when added in very small amounts. In addition, when compared to other hydrophilic compounds, sugars carry the benefits of providing a taste that increases patient compliance and having known biocompatibility due to their use as carriers for decades, when compared to other materials such as polymers or aminoacids whose toxicology to the lung is not as widely studied. Including sugars in inhalation formulations decreases cohesion by adhering to the API and acting as an inert spacer between drug microparticles. Lipids comprise 90% of the surfactant that is present in the lungs, which consists of 40% DPPC by weight and smaller amounts of other lecithins and cholesterol, which provides these materials the recognition of generally safe materials (GRAS) [7]. These compounds protect the drug from humidity and improve aerosolization due to their anti-adherent properties. Cholesterol is a biocompatible material that has been shown to decrease particle aggregation and provide the above-mentioned benefits through drug coating [7][14]. The low melting point of these compounds would hinder their application in techniques such as spray-drying. However, when used in dry co-milling processes combined with sugars, surprisingly these compounds proved to be suitable to provide particles with improved aerodynamic performance (fine particle fraction and emitted dose) and enhanced stability, while decreasing the fouling effect through the process.

The pharmaceutical compositions of the invention comprising crystalline composite particles of API with sugars and lipids produced by co-milling have enhanced performance and stability without hampering dissolution due to the use of wetting agents (sugars) and biocompatible and biodegradable substances that are naturally present in the lung (lipids), while hindering interparticle interactions that cause agglomeration. The particles described in this invention for aerodynamic performance improvement are different from the ones described in the prior art comprising amino-acids, metal stearates or phospholipids, in that we believe the improvement does not come from anti-adherent properties of the excipient, but from the ability of sugar fines to adhere to API active sites and prevent agglomeration by acting as spacers. The particles described in this invention also carry the additional benefit of improving patient compliance through taste.

### GENERAL DESCRIPTION OF FIGURES

FIG. 1 shows the particle size distribution determined by laser diffraction at 5 bar for the trials of Example 1.

FIG. 2 shows the scanning electron micrographs of the co-milled trials of Example 1

FIG. 3 shows the XRPD diffractogram for trials of Example 2.

FIG. 4 shows the XRPD diffractogram for trials of Example 4.

FIG. 5 shows the aerodynamic characterization of the trials of Example 4 using the Plastiape device.

FIG. 6 shows the dissolution profiles of Example 1.

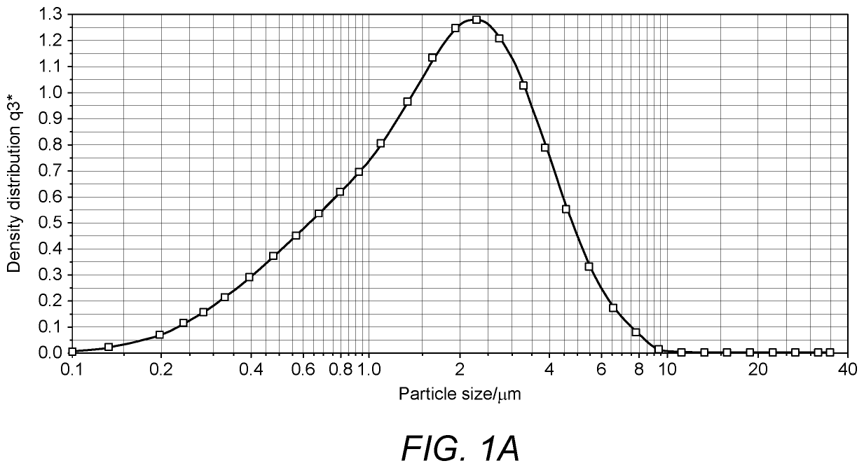

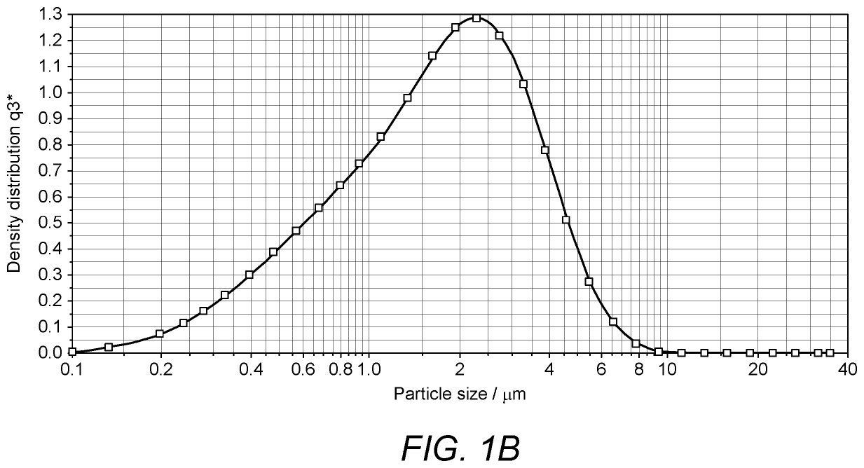

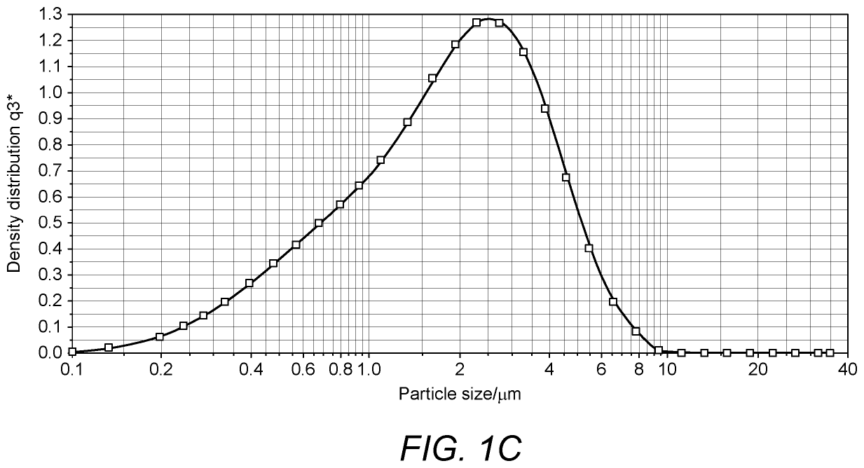

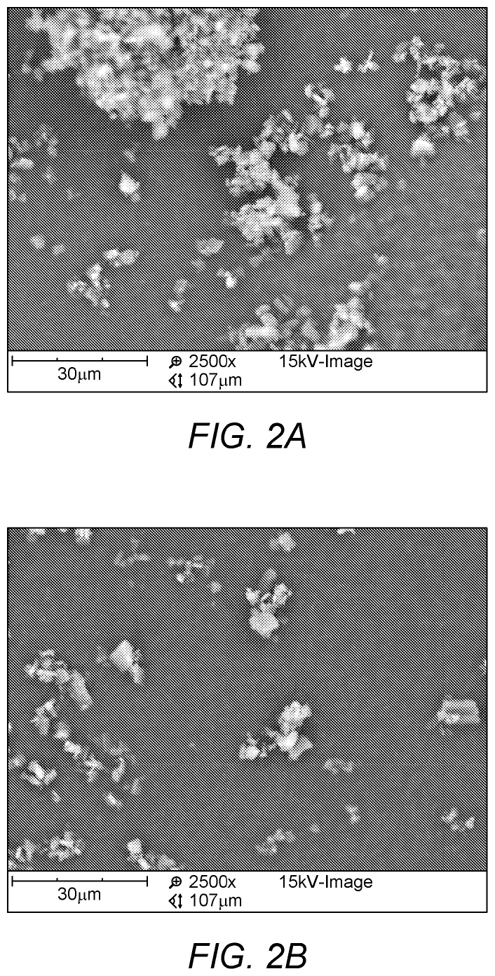

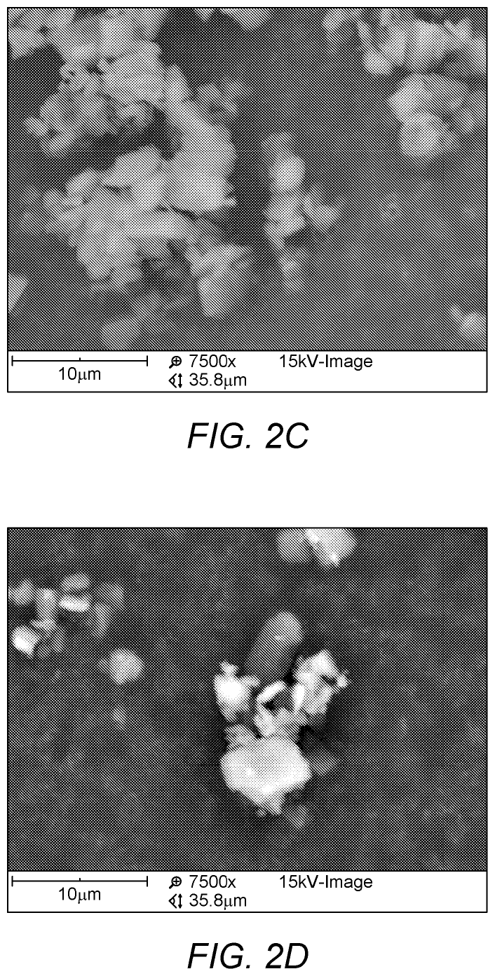

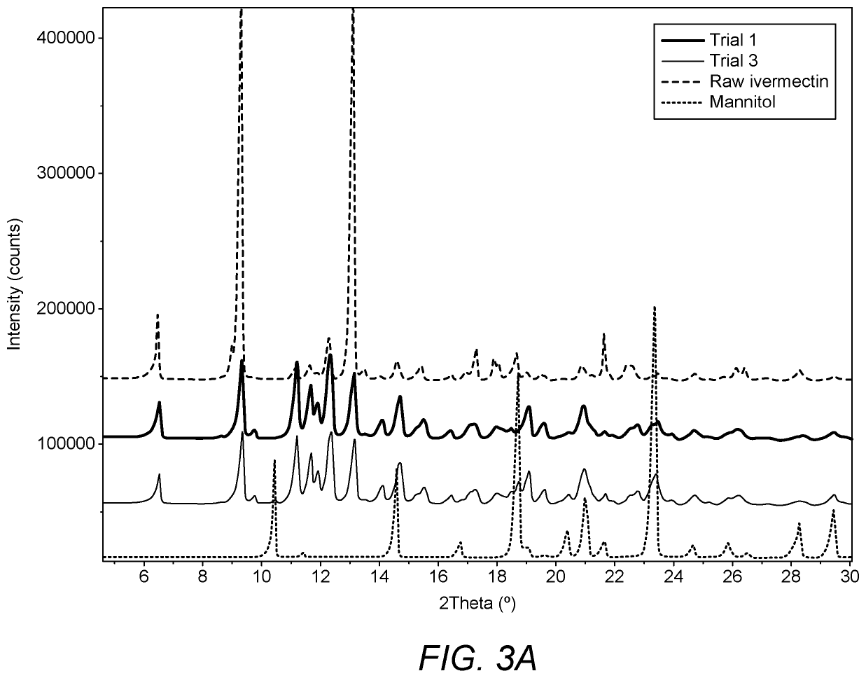

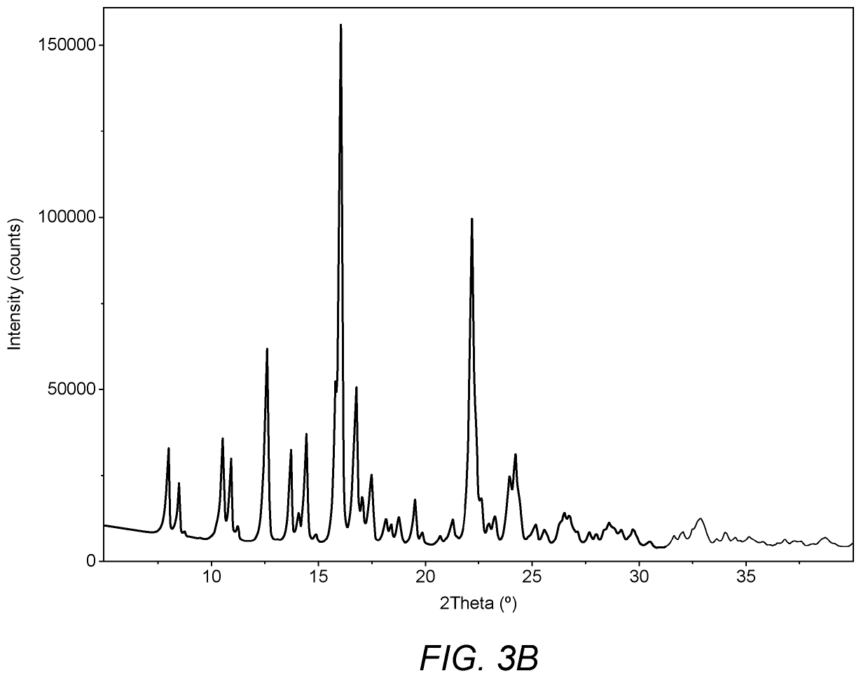

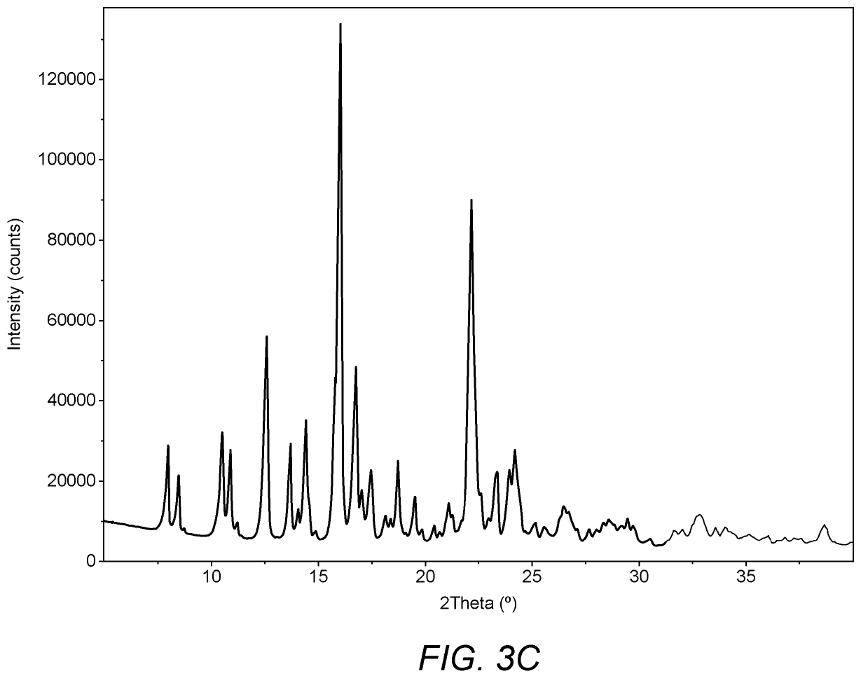

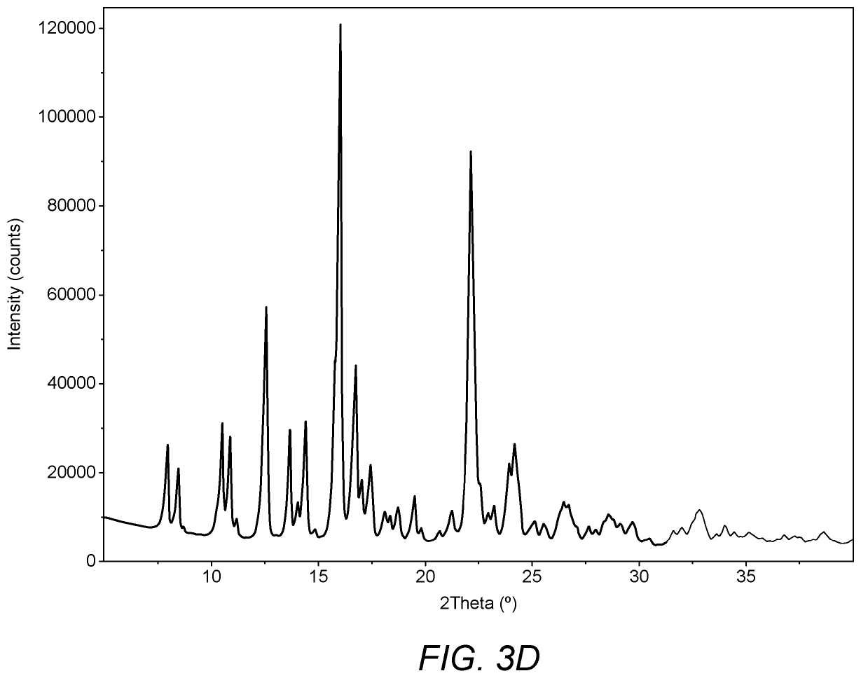

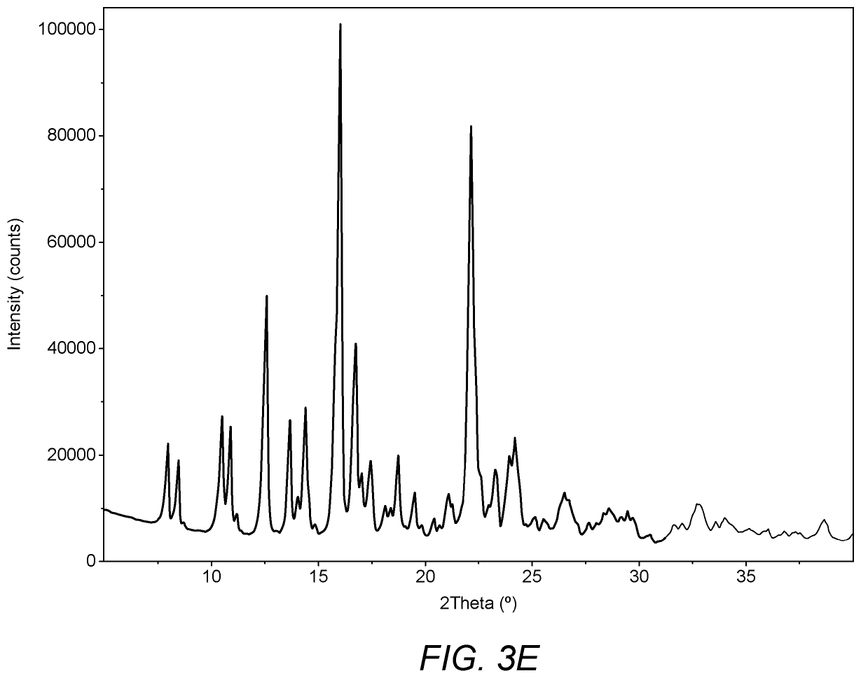

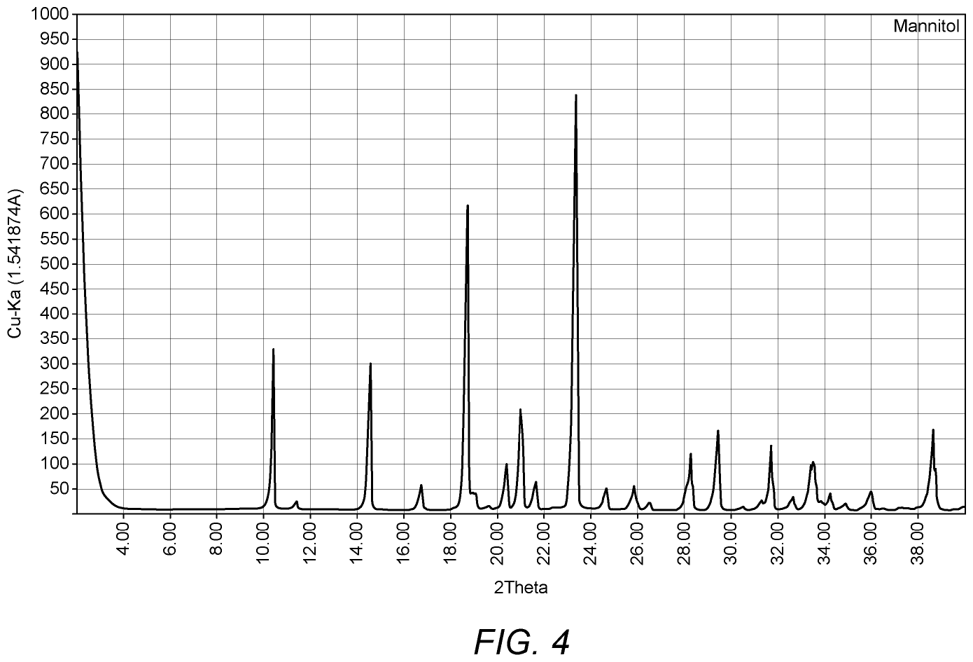

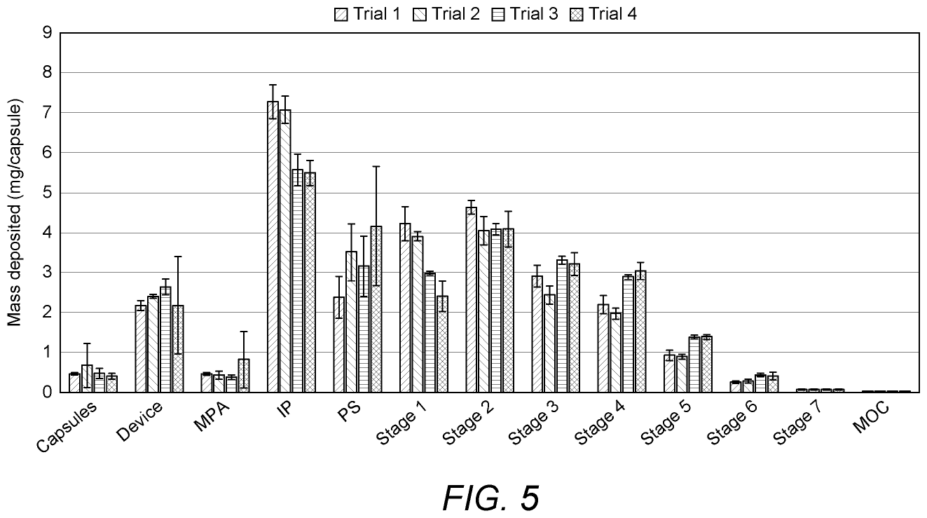

The pharmaceutical composition of the invention preferably comprises composite particles having a particle size distribution which is suitable for inhalation. For example, particle size distribution may be such that the Dv90 is less than or equal to 20 μm. Dv90 is the point in the particle size distribution, up to and including which, 90% of the total volume of material in the sample is ‘contained’. In a preferred aspect, the particle size distribution has a Dv90 of less than or equal to 10 μm.

In one aspect, a pharmaceutical composition according to the invention may have a particle size distribution wherein the range is about 0.1 μm≤Dv90≤6 μm.

The compositions of the invention have been found, when used for example in typical dry powder inhalers, in general to have a greater emitted dose (ED) than other types of composition, for example those which are otherwise similar or the same, but which comprise API alone, or API and only one excipient. Thus, the invention also provides a pharmaceutical composition as described wherein the emitted dose obtained—for example as measured by Dosage Unit Sampling Apparatus (DUSA), or Fast screening impactor (FSI) or Next Generation Impactor (NGI)—is higher than that of a pharmaceutical composition comprising only the API, when the compositions are prepared under the same conditions.

The compositions of the invention have also been found, when used for example in typical dry powder inhalers, in general to have a greater fine particle fraction (FPF) than other types of composition, for example those which are otherwise similar or the same, but which comprise API alone, or comprise API and only one excipient. Thus, the invention also provides a pharmaceutical composition as described wherein the fine particle fraction (FPF) obtained—for example as measured by DUSA, or FSI or NGI—is higher than that of a pharmaceutical composition comprising only the API, when the compositions are prepared under the same conditions.

The compositions of the invention have also been found to have excellent dissolution properties, which is typically better than the dissolution properties of other types of composition, for example those which are otherwise similar or the same, but which comprise API alone, or comprise API and only one excipient. Thus, the invention also provides a pharmaceutical composition as described wherein the dissolution time of the said pharmaceutical composition is decreased when compared with a composition which is the same in all other respects, but which comprises the micronized API alone.

The compositions of the invention have also been found to have good physical and/or chemical stability, which is typically better than the physical and/or chemical stability of other types of composition, for example those which are otherwise similar or the same, but which comprise API alone, or comprise API and only one excipient. Thus, the invention also provides a pharmaceutical composition as described wherein the physical and/or chemical stability of the pharmaceutical composition is increased when compared with a composition comprising the micronized API alone.

Any pharmaceutically acceptable sugar may be used in the compositions of the invention, but especially those which are suitable for use via the inhalation route in human patients. One sugar or a combination of two or more sugars may be used, although preferably a single sugar is employed. Preferably, the sugar is chosen from the group comprising: mannitol, trehalose, trehalose hyclate, sucrose, lactose or raffinose, or a combination of two or more thereof.

In one aspect, pharmaceutical compositions wherein the composite particles comprise a sugar which is mannitol or trehalose, or a combination thereof, are preferred. Mannitol is one particularly preferred sugar. We have found mannitol for example to be advantageous over other sugars approved for inhalation owing to its lower hygroscopicity and nontoxicity. Mannitol is also capable of providing a high fine particle dose of integrated drug upon powder aerosolization.

Any pharmaceutically acceptable lipid may be used in the compositions of the invention, but especially those which are suitable for use via the inhalation route in human patients. One lipid or a combination of two or more lipids may be used, although preferably a single lipid is employed. Preferably, the lipid is chosen from the group comprising: saturated or unsaturated fatty acids; glycerides including neutral glycerides or phosphoglycerides; non-glyceride lipids such as steroids, waxes, or sphingolipids, or a combination of two or more thereof.

In one aspect of the invention, the lipid is chosen from the group comprising a steroid selected from the following steroid classes: cholestanes, cholanes, pregnanes, androstanes, or estanes; or a phosphoglyceride chosen from the group comprising a phosphatidylcholine, a phosphatidylglycerol, or a phosphatidylethanolamine, or a combination of two or more thereof.

In a preferred aspect, the lipid is chosen from the steroid class, in particular the cholestanes such as cholesterol. In a further preferred aspect, the lipid is chosen from the phosphoglyceride or phospholipid group, in particular lipids such as dipalmitoylphosphatidylcholine (DPPC), distearoylphosphatidylcholine (DSPC), dimyristoylphosphatidylcholine (DMPC) or lecithin, or a combination of two or more thereof. Cholesterol and DSPC are two particularly preferred lipids.

Cholesterol is for example the major neutral lipid component found in pulmonary surfactant, and we have found this provides particularly good results when combined with sugar components such as those disclosed herein, including mannitol.

In one aspect, pharmaceutical compositions wherein the composite particles comprise mannitol or trehalose as the sugar, and cholesterol as the lipid are preferred.

A pharmaceutical composition according to the invention comprises a balance of the ingredients in order to provide the desired effects. Preferably, the individual components of the composition are present as follows, wherein the weight % of the components is expressed by weight of the total composition.

The API component is preferably present from 50 to 99.5 wt %, with a preferred range being 80 to 99.5 wt %, depending on the API. A range of 90 to 95 wt % may also be used.

The sugar component is preferably present from 0.5 to 45 wt %, with a preferred range being 0.5 to 20 wt %, depending on the sugar. A preferred sugar such as mannitol may be used to good effect for example in a range of from 5 to 20 wt %, particularly at 10 wt % or more, such as in the range 10 to 20 wt %.

The lipid component is preferably present from 0.01 to 5 wt %, with a preferred range being 0.04 to 2 wt %, depending on the lipid. A preferred lipid such as cholesterol may be used to good effect for example in a range of from 0.04 to 2 wt %.

Accordingly, in a broad aspect, one preferred pharmaceutical composition according to the invention, is wherein the weight % of the components by weight of the total composition range as follows: API from 50 to 99.5 wt %; sugar from 0.5 to 45 wt %; and lipid from 0.01 to 5 wt %.

A further preferred pharmaceutical composition of the invention is wherein the weight % of the components by weight of the total composition range as follows: API from 80 to 99.5 wt %; sugar from 0.5 to 20 wt %; and lipid from 0.04 to 2 wt %.

Some preferred example compositions are shown below in Table 1:

TABLE 1
Examples of compositions according to the invention (ITZ = itraconazole;
IVM = ivermectin; REM = remdesivir)

| Ingredient | ITZ1 | ITZ2 | ITZ3 | IVM1 | IVM2 | ITZ4 | REM1 | REM2 | REM3 |
|---|---|---|---|---|---|---|---|---|---|
| API | 94.5 | 99.5 | 89.76 | 90.58 | 80 | 80 | 89 | 94 | 89 |
| Mannitol | 5 | 0 | 9.31 | 8.66 | 18.18 | 18.14 | 10 | 5 | 10 |
| Cholesterol | 0.5 | 0.04 | 0.93 | 0.76 | 1.82 | 1.86 | 1 | 1 | 1 |
| Trehalose | 0 | 0.46 | 0 | 0 | 0 | 0 | 0 | 0 | 0 |
| TOTAL | 100.00 | 100.00 | 100.00 | 100.00 | 100.00 | 100.00 | 100.00 | 100.00 | 100.00 |

Some preferred ratios (by weight) of API:sugar:lipid range from 89:10:1 to 90:5:5. Examples include 89:10:1, 94:5:1 and 90:5:5. One particularly preferred sugar in these ratios is mannitol, and particularly preferred lipids include cholesterol or DSPC.

In one aspect, the API is present at 30 wt % or more, based on the weight of the total composition, or preferably is present at 50 wt % or more, based on the weight of the total composition.

The API itself can in principle be any API that is suitable for administration using a powder formulation, and in particular one that is suitable for administration via the pulmonary route. The API may for example be an antifungal agent such as itraconazole; an antiparasitic drug such as ivermectin; or an antiviral drug such as remdesivir.

The present invention is of utility in enabling high dosages of API to be provided, particular via the inhalation route. Thus, the invention provides a pharmaceutical composition as described, wherein the composition is a high dosage inhalation composition wherein a single inhaled dose provides at least 2.5 mg of API or more, such as greater than 5 mg or more. High dosage can also refer to where the amount of API in the inhaled drug dose is above 4% by weight of the dose (see for example [20] Sibum et al, 2018; Adhikari et al, 2022).

With regard to inhalers, different types of inhaler may be employed for use with the composition of the present invention but a dry powder inhaler is preferred and may for example be a single use inhaler. Preferably, the dry powder inhaler comprises a mouthpiece, an inhaler body and a cartridge for receiving the dose, as will be understood. In a preferred aspect, the cartridge is moveable in relation to an inhaler body, for making a dose available through a mouthpiece. The dry powder inhaler employed may comprise one wherein the inhaler cartridge comprises one reservoir or multiple reservoirs. In one preferred aspect, each reservoir of the inhaler cartridge provides a single dose.

As noted above, in a broad, a process according to the invention comprises the steps of:
a. Blending API and one or more excipients comprising at least one sugar or at least one lipid, or both at least one sugar and at least one lipid, into a homogeneous powder;
b. Reducing the particle size distribution of the blend.

Thus, in one aspect, step (a) may comprise blending API and sugar, followed by step (b) for the blend of API and sugar. In this case, the process will involve a further step (c) wherein the blend of API and sugar which has undergone step (b) is further blended with a lipid component, and this resulting blend is then subjected to a second step (b)—that is, reducing the particle size distribution of the blend of API and sugar, together with the lipid component. In a preferred aspect, step (c) is a co-milling step, for example co-milling of API plus sugar and lipid byjet-milling. Thus, the process of the invention always includes at least one step, preferably a co-milling step, wherein at least one sugar and at least one lipid, together with the API, are together subjected to a step of reducing the particle size distribution of the blend.

Thus, in the process of the invention as disclosed and claimed herein, in one preferred aspect, the process may comprise wherein the API and at least one sugar are first blended and co-milled together, for example by jet-milling, and at least one lipid is then blended and jet-milled with the resulting pharmaceutical composition, yielding a pharmaceutical composition comprising API, at least one sugar and at least one lipid.

The process of the invention thus encompasses the possibility of two co-milling steps, if desired. A first co-milling step of API with a sugar component alone (or lipid component alone), and a second co-milling step wherein a blend of API with a sugar component (or a blend of API with a lipid component), is co-milled with a second component which is not yet present i.e. either the lipid component, or the sugar component, as appropriate.

A feature of the present invention is thus that both excipient components (i.e. sugar and lipid) are subjected to a step (b), preferably by co-milling, with the API. This is preferably by jet milling. This helps enable the provision of improved aerodynamic performance. Another preferred feature of the process is that no second blending step is required, after co-milling of the API, sugar and lipid.

Step (a) of the process may comprise simultaneous blending of the API with the at least one sugar and the at least one lipid, or may comprise sequential blending of the at least one sugar and the at least one lipid. That is, the blending of the sugar and lipid components may be done one after the other. The order of blending is not critical, thus for sequential blending, either the sugar or the lipid may blended first with the API, followed by the second component.

The injection pressure on the jet milling step may for example preferably be between about 2 and about 12 bar. In one aspect, the injection pressure ranges from about 4 and about 8 bar.

The temperature at which the size reduction step (step (b) is carried out is preferably at about 60° C. or below.

In some aspects, the temperature at which the size reduction step (step (b) is carried out is at about 10° C. or below. For example, this step may be done as a cryo-jet milling step, which is jet milling under cold conditions, primarily to avoid amorphous form formation.

In other aspects, the temperature at which the size reduction step (step (b) is carried out is at about 20° C. or above. Thus, a temperature of between 20° C. and 60° C. is often suitable.

In the process of the invention, one advantage is that it is possible to dispense with a conditioning step. In a typical conditioning step, formulations during their manufacture are stored under controlled conditions of temperature and relative humidity. Thus, in a preferred aspect, a process as disclosed and claimed herein comprises wherein no conditioning step is employed to produce the final ready to use formulation, or wherein a reduced amount of conditioning time is employed when compared with the conditioning time required to condition a composition comprising micronized API alone.

### DETAILED DESCRIPTION OF THE INVENTION

In one embodiment, the invention can be carried out by blending in a low-shear mixer previously sieved (600 μm) API particles with, for example, previously sieved (600 μm) mannitol particles and cholesterol particles at, for example, 96 rpm for 10 minutes. The resulting composition being milled, for example, in a vertical jet-mill using compressed nitrogen to generate a grinding pressure of, for example, 6 bar and a Venturi pressure of 7 bar, the composition being fed at, for example, 40 g/h to the jet-mill apparatus. The jet-mill is the simplest dry size reduction apparatus, comprising a milling chamber (usually flat disk shaped with grinding nozzles) where the raw material is fed through a Venturi, compressed gas (usually air or nitrogen) being used to generate the injection pressure and grinding pressure through the nozzles, generating a vortex which promotes particle-particle and particle-wall collisions that induce fracture and subsequent size reduction. The obtained particle size distribution is manipulated by varying applied grinding pressure or the solids flowrate and the Venturi pressure is defined usually 1 bar above, to prevent backflow. An amount, for example, between 20 and 50 mg of the resulting composition is filled to, for example, size 3 HPMC capsules which are ready to be actuated using a DPI device.

Different aerodynamic particle sizes can be produced according to the target respiratory tract region. Varying the employed specific energy in the milling process allows different particle sizes to be obtained. Carrying out co-jet-milling at grinding pressures in the region of 2-6 bar leads to a particle size distribution reduction to inhalable sizes for some APIs, but a pressure of 6-12 bar is required for harder ones. The temperature at which the process is carried out affects particle mobility, thereby influencing the brittle and ductile regions of the material. For some materials, co-milling in the temperature range of 0-20° C. can be required to provide enough brittleness. For other materials, co-milling at temperatures of 20-60° C. is required so that adhesion of additive particles to active particles occurs. Wet methods such as high pressure homogenization can also be used to perform co-milling where desired. This method is is for example able to provide round particles, contrarily to the jet-mill where particle shape is not controlled, in addition to yielding a narrower particle size distribution. Co-milling with certain excipients in the weight percentage of 0.5% has been reported to significantly increase the aerodynamic performance [9]. On the other hand, additive amounts of up to 20% are beneficial when dealing with certain active particles.

Formulations in the examples of the present invention were processed using:
1) The Turbula (Willy A. Bachofen A G, Basel, Switzerland) low shear mixing apparatus.
2) The laboratory scale jet-mill MCOne (Jetpharma Solutions S A, Balerna, Switzerland)

All materials except cholesterol were sieved manually at a size specified in each example. Blending and milling conditions are also specified in each example, these conditions are based on the applicant's previous milling experiments.

Formulations in the examples of the present invention comprised one of more of the following materials:
Lactose Respitose SV003 from DFE Pharma, Germany
Mannitol Pearlitol from Roquet, USA
Trehalose dihydrate from Sigma Aldrich, USA
Cholesterol from Sigma Aldrich, USA
Itraconazole from Fagron Iberica, Spain
Ivermectin from Hovione PharmaScience, Portugal
Remdesivir from Hangzhou MolCore BioPharmatech Co., Ltd

Formulations in the examples of the present invention were characterized by the following techniques:
A Helos laser diffraction instrument, combined with a Rodos dry dispersing unit and an Aspiros module (Sympatec GmbH, Germany) were used for particle size distribution measurements for most formulations. Dispersing pressures of 0.1 bar (using an R2 lens (0.45-87.5 μm), with a focal length of 50 mm) and 5 bar (using an R1 lens (0.18-35 μm), with a focal length of 20 mm) were applied in order to determine the size of either agglomerates or single particles, respectively. The velocity was maintained at 50 mm/s. All measurements were done in duplicate.
Hydroxypropylmethyl cellulose (HPMC) size three capsules (Capsugel, Colmar, France) containing 30 mg±1.5 mg of powder were used for all in-vitro aerosolization studies. All were tested with two actuations of the same capsule in the Fast-Screening Impactor (FSI) (Copley Scientific, Nottingham, UK) with 15 ml of dissolution media in the pre-separator, which was connected to a vacuum pump (Copley Scientific, Nottingham, UK), at either 60 L/min or 100 L/min flowrate. Quantification of the emitted dose (ED) was carried out gravimetrically by weighing the inhaler device and capsule before and after actuation, and the fine particle dose (FPD) was measured gravimetrically by weighing the filter before and after actuation. The cutoff size of the pre-separator was 5 μm, thereby the fraction of the label claimed mass that reached the filter (the fine particle dose) was the fine particle fraction (FPF). The ED comprised all of the API beyond the inhaler device. Some formulations were characterized in the Next Generation Impactor (NGI) (Copley Scientific, Nottingham, UK) with a pre-separator connected to a vacuum pump (Copley Scientific, Nottingham, UK). The NGI cups were coated with 1 mL of 1% of glycerol in ethanol (v/v) solution. 15 ml of dissolution media were placed in the pre-separator. Each test consisted of one actuation of the capsule into the NGI using either a 60 L/min or a 100 L/min DPI device, during 4 s or 2.4 s, respectively. The tests were performed in triplicate. API content deposited in each stage was recovered and analyzed by HPLC, enabling ED and FPD determination and the distribution among stages, assuring a mass balance of the recovered material with an error below 15%. All aerodynamic performance experiments were carried out in triplicate.
X-ray powder diffraction (XRPD) patterns were obtained with a PANalytical (Malvern, UK) X'Pert PRO X-ray diffraction system using Cu K radiation (λ=1.54 A°). The generator voltage and current intensity were set at 45 kV and 40 mA, respectively, and the 2 θ scanning range was from 4° to 40° with a step size 0.0131303° and a count time of 99.450 s per step. Samples were loaded using the zero-background technique.

### Example 1—Co-Milling Itraconazole with Sugar and Lipid

Trials 1, 2, 3, 4 and 5 were prepared by low shear mixing and jet-milling.

Trial 1: Mannitol previously sieved with a 600 μm sieve and cholesterol were blended in a low shear mixer at 96 rpm for 10 min. The previous blend was blended with previously sieved with a 600 μm sieve itraconazole in a low shear mixer at 96 rpm for 10 min. This ternary blend was fed to a lab scale vertical jet-mill and submitted to the conditions described in Table 1.2.

Trial 2: Mannitol previously sieved with a 600 μm sieve was blended with previously sieved with a 600 μm sieve itraconazole in a low shear mixer at 96 rpm for 10 min. This blend was fed to a lab scale vertical jet-mill submitted conditions described in Table 1.2.

Trial 3: (API alone): Itraconazole was sieved with a 600 μm sieve and fed to a lab scale vertical jet-mill and submitted to the conditions described in Table 1.2.

Trial 4: Trehalose dihydrate previously sieved with a 600 μm sieve and cholesterol were blended in a low shear mixer at 96 rpm for 10 min. The previous blend was blended with previously sieved with a 600 μm sieve itraconazole in a low shear mixer at 96 rpm for 10 min. This ternary blend was fed to a lab scale vertical jet-mill and submitted to the conditions described in Table 1.2.

Trial 5: Mannitol previously sieved with a 600 μm sieve and cholesterol were blended in a low shear mixer at 96 rpm for 10 min. The previous blend was blended with previously sieved with a 600 μm sieve itraconazole in a low shear mixer at 96 rpm for 10 min. This ternary blend was fed to a lab scale vertical jet-mill and submitted to the conditions described in Table 1.2.

TABLE 1.2
Summary of the process parameters and formulation compositions of Example 1.

| | Griding | Venturi | Feed | | Trehalose | | |
|---|---|---|---|---|---|---|---|
| | pressure | pressure | flow rate | API | dihydrate | Mannitol | Cholesterol |
| Trial | (bar) | (bar) | (g/h) | (g) | (g) | (g) | (g) |
| 1 | 6.0 | 7.0 | 40 | 6.615 | 0 | 0.350 | 0.035 |
| 2 | 6.0 | 7.0 | 40 | 6.650 | 0 | 0.350 | 0 |
| 3 | 6.5 | 7.5 | 40 | 10.000 | 0 | 0 | 0 |
| 4 | 6.5 | 7.5 | 40 | 6.965 | 0.032 | 0 | 0.003 |
| 5 | 6.5 | 7.5 | 40 | 6.283 | 0 | 0.652 | 0.065 |

The micronized material was characterized for particle size distribution by laser diffraction, crystalline state by XRPD and morphology by SEM—summarized in

Table 2, Error! Reference source not found. and Error! Reference source not found . . .

All trials present a particle size within the inhalation range. Error! Reference source not found . . . A (particle size distribution of Trial 3) and Error! Reference source not found . . . B (particle size distribution of Trial 5) show only one (similar) population of particles indicating that either composite particles were formed or the API and excipients were micronized to a similar extent.

The milled material presented an XRPD identical to the one of the API alone (Trial 3) or with mannitol (Trials 1, 2 and 5) or trehalose dihydrate (Trial 4) diffraction peaks, indicating there is no change in the solid state of the materials.

SEM micrographs of Trial 3, API alone, and Trial 5, co-milled material with mannitol and cholesterol, indicate the presence of excipients do not have a clear impact on particle morphology.

TABLE 2
Micronized blends characterization for Example 1.

| | Trial 1 | Trial 2 | Trial 3 | Trial 4 | Trial 5 |
|---|---|---|---|---|---|
| Dv10 | 0.5 | 0.6 | 0.5 | 0.5 | 0.5 |
| (μm) | | | | | |
| Dv50 | 1.7 | 1.9 | 1.8 | 1.5 | 1.8 |
| (μm) | | | | | |
| Dv90 | 3.9 | 4.3 | 3.9 | 3.3 | 3.8 |
| (μm) | | | | | |
| SPAN | 1.9 | 1.9 | 1.9 | 1.9 | 1.9 |
| XRPD | Conforms | Conforms | Conforms | Conforms | Conforms |
| | to the | to the | to the | to the | to the |
| | reference | reference | reference | reference | reference |
| | diffractogram | diffractogram | diffractogram | diffractogram | diffractogram |
| | with | with | | with | with |
| | mannitol | mannitol | | trehalose | mannitol |
| | peaks | peaks | | dihydrate | peaks |
| | | | | peaks | |

Following micronization, the powder obtained from each trial was filled into HPMC size 3 capsules, at 20-25° C. and 50±10% RH, with a fill-weight of 30 mg. Each capsule was actuated using a Plastiape inhaler (flow rate of 100 L/min for a pressure drop of 4 kPa).

The manufactured capsules were characterized for aerodynamic performance by FSI, summarized in Table 3.

The results of Trials 1, 2 and 3 show a fine particle fraction improvement of from 39.9±0.1% of emitted dose for the API alone (Trial 3) to 50±1.2% of emitted dose for Trial 2 containing API and mannitol, and to 59.4±0.9% of emitted dose for Trial 1 containing API, mannitol, and cholesterol. These results prove that the pharmaceutical composition comprising a sugar and cholesterol, produced by co-milling, significantly improves the aerodynamic performance in comparison to co-milling with a sugar only. The results of Trials 3, 4 and 5 show a fine particle fraction (FPF) improvement of from 39.9±0.1% of emitted dose for the API alone (Trial 3) to 55.7±0.5% of emitted dose for Trial 4 containing API, trehalose dihydrate and cholesterol, and to 56.5±1.1% of emitted dose for Trial 5 containing API, mannitol, and cholesterol.

These results show that the improvements attained by the pharmaceutical composition comprising a sugar and cholesterol, produced by co-milling, improve the aerodynamic performance through a different range of weight percentage of excipient (0.5 wt % of excipient for Trial 4, 5 wt % of excipients for Trial 1, and 10.3 wt % of excipients for Trial 5).

TABLE 3
Summary of the capsule characterization for Example 1.

| | Trial 1 | Trial 2 | Trial 3 | Trial 4 | Trial 5 |
|---|---|---|---|---|---|
| Fill weight | 30 ± 1.5 | 30 ± 1.5 | 30 ± 1.5 | 30 ± 1.5 | 30 ± 1.5 |
| (mg) | | | | | |
| Device used | Plastiape | Plastiape | Plastiape | Plastiape | Plastiape |
| | (100 L/min) | (100 L/min) | (100 L/min) | (100 L/min) | (100 L/min) |
| ED | 29.6 ± 0.0 | 28.0 ± 0.0 | 27.4 ± 0.2 | 27.9 ± 0.6 | 27.9 ± 0.1 |
| (mg/capsule) | | | | | |
| FPD | 16.4 ± 0.3 | 14.0 ± 0.4 | 11.0 ± 0.1 | 15.5 ± 0.5 | 15.8 ± 0.3 |
| (mg/capsule) | | | | | |
| FPF_{ED} (%) | 59.4 ± 0.9 | 50 ± 1.2 | 39.9 ± 0.1 | 55.7 ± 0.5 | 56.5 ± 1.1 |

### Example 2—Co-Milling Ivermectin with Sugar and Lipid

Trials 2 and 3 were prepared by low shear mixing and jet-milling.

Trial 1 (API alone): Ivermectin was sieved with a 600 μm sieve and fed to a lab scale vertical jet-mill and submitted to the conditions described in Table 4.

Trial 2: Mannitol previously sieved with a 600 μm sieve and Cholesterol were blended in a low shear mixer at 96 rpm for 10 min. The previous blend was blended with previously sieved with a 600 μm sieve Ivermectin in a low shear mixer at 96 rpm for 10 min. This ternary blend was fed to a lab scale vertical jet-mill and submitted to the conditions described in Table 4

Trial 3: Mannitol previously sieved with a 600 μm sieve and Cholesterol were blended in a low shear mixer at 96 rpm for 10 min. The previous blend was blended with previously sieved with a 600 μm sieve Ivermectin in a low shear mixer at 96 rpm for 10 min. This ternary blend was fed to a lab scale vertical jet-mill and submitted to the conditions described in Table 4.

TABLE 4
Summary of the process parameters and
formulation compositions of Example 2.

| | Griding | Venturi | Feed | | | |
|---|---|---|---|---|---|---|
| | pressure | pressure | flow rate | API | Mannitol | Cholesterol |
| Trial | (bar) | (bar) | (g/h) | (g) | (g) | (g) |
| 1 | 6.5 | 7.5 | 20 | 5.000 | 0.000 | 0.000 |
| 2 | 6.5 | 7.5 | 20 | 4.875 | 0.466 | 0.041 |
| 3 | 6.5 | 7.5 | 20 | 4.000 | 0.909 | 0.091 |

The micronized material was characterized for particle size distribution by laser diffraction, and crystalline state by XRPD—summarized in

Table 5 and Error! Reference source not found . . . All trials present a particle size within the inhalation range, with the trial 3 (lower API content, higher mannitol, and cholesterol content) presenting a smaller particle size. These results indicate the presence of the two excipients may facilitate particle breakage, and therefore lead to a smaller PSD for similar specific micronization energies. The milled material presented an XRPD identical to the one of the API or with mannitol diffraction peaks for Trials 2 and 3, indicating the API and excipient are crystalline after micronization.

TABLE 5
Micronized blends product characterization for Example 2.

| | Trial 1 | Trial 2 | Trial 3 |
|---|---|---|---|
| Dv10 (μm) | 0.6 | 0.6 | 0.6 |
| Dv50 (μm) | 2.2 | 2.2 | 1.8 |
| Dv90 (μm) | 5.4 | 5.2 | 4.3 |
| SPAN | 2.1 | 2.1 | 2.0 |
| XRPD | Conforms to the | Conforms to the | Conforms to the |
| | reference | reference | reference |
| | diffractogram | diffractogram with | diffractogram with |
| | | mannitol peaks | mannitol peaks |

Following micronization, the powder obtained from Trials 1, 2 and 3 were manually filled into HPMC size 3 capsules, at 20-25° C. and 50±10% RH, with a target fill-weight of 30 mg, and actuated using a Plastiape inhaler (flow rate of 100 L/min for a pressure drop of 4 kPa) to assess aerodynamic performance.

The filled capsules have a fine particle fraction (determined by gravimetric FSI) of 14.1±2.1% of emitted dose for the API alone, and 31.6±0.5% and 41.9±1.4% of emitted dose, for Trial 2 and 3 containing API, mannitol and cholesterol in the weight percentage of 9.4 and 20.0%, respectively—see

Table 6. The results indicated that the pharmaceutical composition comprising a sugar and cholesterol, produced by co-milling, had a beneficial impact for the aerodynamic performance of a high dosage ivermectin formulation, in both the emitted dose and fine particle dose. Its impact increasing consecutively as more excipient was added, leading to an FPF almost 3 times larger, compared with the formulation milled without excipient.

TABLE 6
Summary of the capsule characterization for Example 2.

| | Trial 1 | Trial 2 | Trial 3 |
|---|---|---|---|
| Fill weight (mg) | 30.0 ± 1.5 | 30.0 ± 1.5 | 30.0 ± 1.5 |
| FPD (mg powder/caps) | 5.5 ± 0.1 | 9.1 ± 0.1 | 12.2 ± 0.4 |
| ED (mg powder/caps) | 26.4 ± 1.9 | 28.9 ± 0.8 | 29.1 ± 0.1 |
| FPF (% of ED) | 14.1 ± 2.1 | 31.6 ± 0.5 | 41.9 ± 1.4 |

### Example 3—Conditioning a Co-Milled High Dosage Formulation of Itraconazole

Trials 2 and 3 were prepared by low shear mixing and jet-milling.

Trial 1 (API alone): Itraconazole was sieved with a 600 μm sieve and fed to a lab scale vertical jet-mill and submitted to the conditions described in Table 7.

Trial 2: Trehalose dihydrate previously sieved with a 600 μm sieve and Cholesterol were blended in a low shear mixer at 96 rpm for 10 min. The previous blend was blended with previously sieved with a 600 μm sieve Itraconazole in a low shear mixer at 96 rpm for 10 min. This ternary blend was fed to a lab scale vertical jet-mill and submitted to the conditions described in Table 7.

Trial 3: Mannitol previously sieved with a 600 μm sieve and Cholesterol were blended in a low shear mixer at 96 rpm for 10 min. The previous blend was blended with previously sieved with a 600 μm sieve Itraconazole in a low shear mixer at 96 rpm for 10 min. This temary blend was fed to a lab scale vertical jet-mill and submitted to the conditions described in Table 7.

For stability assessment, 2 g of the powder obtained from Trials 1, 2 and 3 were stored in hermetic conditions in an oven at 40° C. and 75±5% RH for 4 weeks, herein referred to as “stability assessment samples”.

TABLE 7
Summary of the process parameters and formulation compositions of Example 3.

| | Griding | Venturi | Feed | | | Trehalose | |
|---|---|---|---|---|---|---|---|
| | pressure | pressure | flow rate | API | Mannitol | dihydrate | Cholesterol |
| Trial | (bar) | (bar) | (g/h) | (g) | (g) | (g) | (g) |
| 1 | 6.5 | 7.5 | 40 | 7.00 | 0 | 0 | 0 |
| 2 | 6.5 | 7.5 | 40 | 5.60 | 0 | 1.27 | 0.13 |
| 3 | 6.5 | 7.5 | 40 | 5.60 | 1.27 | 0 | 0.13 |

The micronized material was characterized for particle size distribution by laser diffraction—summarized in Table 7. All trials present a particle size within the inhalation range.

TABLE 7
Micronized blends product characterization for Example 3.

| | | |
|---|---|---|
| Trial 1 | Trial 2 | Trial 3 |

| Timepoint | T0 | T2W | T0 | T2W | T0 | T2W |
|---|---|---|---|---|---|---|
| Dv10 (μm) | 0.5 | 0.5 | 0.5 | 0.5 | 0.5 | 0.6 |
| Dv50 (μm) | 1.8 | 1.8 | 1.7 | 1.7 | 1.8 | 1.8 |
| Dv90 (μm) | 3.9 | 3.9 | 3.7 | 3.8 | 4.0 | 3.9 |
| SPAN | 1.9 | 1.9 | 1.9 | 1.9 | 1.9 | 1.9 |

The micronized product obtained from Trials 1, 2 and 3 and the stability assessment samples of the same trials were filled into HPMC size 3 capsules, at 20-25° C. and 50±10% RH, with a fill-weight of 30 mg, and actuated using a Plastiape inhaler (flow rate of 100 L/min for a pressure drop of 4 kPa).

The filled capsules have a fine particle fraction (determined by FSI) of 39.9±0.1% of emitted dose for the API alone, and 54.7±1.4 and 52.0±0.2% of emitted dose, for Trial 2 and 3 containing API, trehalose dihydrate/mannitol and cholesterol in the weight percentage of 20.0%, respectively—see Table 8. The filled capsules obtained from stability assessment samples have a fine particle fraction (determined by FSI) of 53.5±0.4% of emitted dose for the API alone, and 52.4±1.4 and 51.6±1.0% of emitted dose, for Trial 2 and 3 containing API, trehalose dihydrate/mannitol and cholesterol in the weight percentage of 20.0%, respectively—see Table 8. This indicated the pharmaceutical composition comprising a sugar and cholesterol, produced by co-milling, enabled the powder conditioning step that is generally required after jet-milling to be omitted, for the high dosage Itraconazole formulations, as formulations of said pharmaceutical composition performed similarly in terms of FPF after 4 weeks in accelerated stability conditions (varying only 4% and 1% for Trials 2 and 3, respectively) while the formulation milled without excipient exhibited a 34% variation, thereby requiring a conditioning period until final performance is achieved.

TABLE 8
Summary of the capsule characterization for Example 3.

| | Timepoint | Trial 1 | Trial 2 | Trial 3 |
|---|---|---|---|---|
| ED (mg) | 0 | 27.4 ± 0.2 | 27.6 ± 1.3 | 29.1 ± 0.5 |
| | 4 weeks | 28.4 ± 0.1 | 27.7 ± 0.1 | 29.0 ± 0.6 |
| FPD (mg) | 0 | 11.0 ± 0.1 | 15.0 ± 0.3 | 15.1 ± 0.3 |
| | 4 weeks | 15.2 ± 0.2 | 14.5 ± 0.4 | 15.0 ± 0.6 |
| FPF (% | 0 | 39.9 ± 0.1 | 54.7 ± 1.4 | 52.0 ± 0.2 |
| of ED) | 4 weeks | 53.5 ± 0.4 | 52.4 ± 1.4 | 51.6 ± 1.0 |

### Example 4—Co-Milling Remdesivir with Sugar and Lipid

Trials 2, 3 and 4 were prepared by low shear mixing and jet-milling.

Trial 1 (API alone): Remdesivir was sieved with a 450 μm sieve and fed to a lab scale vertical jet-mill and submitted to the conditions described in Table 8.

Trial 2: Mannitol previously sieved with a 450 μm sieve was blended with previously sieved with a 450 μm sieve remdesivir in a low shear mixer at 96 rpm for 15 min. The previous blend was fed to a lab scale vertical jet-mill and submitted to the conditions described in Table 8.

Trial 3: Cholesterol was blended with previously sieved with a 450 μm sieve remdesivir in a low shear mixer at 96 rpm for 15 min. The previous blend was fed to a lab scale vertical jet-mill and submitted to the conditions described in Table 8.

Trial 4: Mannitol previously sieved with a 450 μm sieve and cholesterol were blended in a low shear mixer at 96 rpm for 15 min. The previous blend was blended with previously sieved with a 450 μm sieve remdesivir in a low shear mixer at 96 rpm for 15 min. This ternary blend was fed to a lab scale vertical jet-mill and submitted to the conditions described in Table 8.

TABLE 8
Summary of the process parameters and
formulation composition for Example 4.

| | Griding | Venturi | Feed | | | |
|---|---|---|---|---|---|---|
| | pressure | pressure | rate flow | API | Mannitol | Cholesterol |
| Trial | (bar) | (bar) | (g/h)² | (g) | (g) | (g) |
| 1 | 4.5 | 6 | 15 | 10 | 0 | 0 |
| 2 | 4.5 | 6 | 15 | 9 | 1 | 0 |
| 3 | 4.5 | 6 | 15 | 9.9 | 0 | 0.1 |
| 4 | 4.5 | 6 | 15 | 8.9 | 1 | 0.1 |

The micronized material was characterized for particle size distribution by laser diffraction, and crystalline state by XRPD—summarized in

Table 9 and Error! Reference source not found . . . All trials present a particle size within the inhalation range, with the trial 4 (cholesterol and mannitol) presenting a smaller particle size. These results indicate the presence of the two excipients may facilitate particle breakage, and therefore lead to a smaller PSD for similar specific micronization energies. The milled material presented an XRPD identical to the one of the API or with mannitol diffraction peaks for Trials 2 and 3, indicating the API and excipient are crystalline after micronization.

TABLE 9
Micronized blends characterization for Example 4.

| | Trial 1 | Trial 2 | Trial 3 | Trial 4 |
|---|---|---|---|---|
| Dv10 (μm) | 0.6 | 0.6 | 0.5 | 0.5 |
| Dv50 (μm) | 2.3 | 2.3 | 2.0 | 1.8 |
| Dv90 (μm) | 6.0 | 6.3 | 5.8 | 5.4 |
| SPAN | 2.4 | 2.4 | 2.6 | 2.8 |
| XRPD | Conforms to the | Conforms to the | Conforms to the | Conforms to the |
| | reference | reference | reference | reference |
| | diffractogram | diffractogram | diffractogram | diffractogram |
| | | with mannitol | | with mannitol |
| | | peaks | | peaks |

Following micronization, the powder obtained from Trials 1, 2, 3 and 4 was filled into HPMC size 3 capsules, using the Auger filling Quantos equipment at 20-25° C. and 50±10% RH, targeting a label claim of 30 mg and a rejection limit of ±5%, and actuated using a Plastiape inhaler (flow rate of 100 L/min for a pressure drop of 4 kPa).

The manufactured capsules were characterized for aerodynamic performance by NGI, summarized in Table 10.

The filled capsules present a significantly different performance in the presence of cholesterol, quantified by the difference in FPF—Error! Reference source not found.: For trials 1 and 2 (API alone and API and mannitol, respectively), the obtained FPF are 37.0±3.8% and 33.4±1.8%, respectively, with no impact of the presence of mannitol. For trials 3 and 4 (API and cholesterol, and API, mannitol and cholesterol, respectively), the FPF are 44.7±1.4% and 44.0±3.9%, respectively, thus approximately 20% higher than the previous trials. These results indicate the presence of additives, in particular cholesterol, to the jet milling process as described in the present invention, leads to a significant performance increase.

TABLE 10
Summary of the capsule characterization for Example 4.

| | Trial 1 | Trial 2 | Trial 3 | Trial 4 |
|---|---|---|---|---|
| Fill weight (mg) | 30.0 ± 1.5 | 33.3 ± 1.5 | 30.3 ± 1.5 | 33.7 ± 1.5 |
| Label claim (mg) | 30 | 30 | 30 | 30 |
| Device used | Plastiape | Plastiape | Plastiape | Plastiape |
| | (100 L/min) | (100 L/min) | (100 L/min) | (100 L/min) |
| ED (mg/capsule) | 25.3 ± 0.4 | 24.7 ± 0.4 | 24.3 ± 0.1 | 25.2 ± 0.7 |
| FPD | 9.4 ± 0.8 | 8.2 ± 0.5 | 10.9 ± 0.4 | 11.1 ± 0.7 |
| (mg/capsule) | | | | |
| FPF_{ED} (%) | 37.0 ± 3.8 | 33.4 ± 1.8 | 44.7 ± 1.4 | 44.0 ± 3.9 |
| MMAD (μm) | 4.1 ± 0.3 | 4.1 | 3.2 ± 0.1 | 3.0 ± 0.2 |
| GSD | 2.2 | 2.3 | 2.2 | 2.1 ± 0.1 |

### Example 5—Pharmaceutical Formulation Containing Mannitol and Cholesterol with Low Flow-Rate Device

A pharmaceutical formulation as described in Example 4 (co-milled remdesivir filled into capsules with label claim of 30 mg, trials 1 to 4) were actuated using a PowdAir inhaler (flow rate of 60 L/min for a pressure drop of 4 kPa), and the results are presented in Table 11.

The filled capsules have an emitted dose of 3.6±3.7 mg for the API alone, 4.9±5.4 mg and 17.2±14.4 mg for Trials 2 and 3, containing mannitol or cholesterol, respectively, and 26.0±1.7 mg for Trial 4, containing cholesterol and mannitol. Thus, the pharmaceutical composition comprising a sugar and cholesterol in a ratio of 10:1 and an API concentration of 89%, produced by co-milling, leads to an ED improvement of 500%, compared with the API alone formulation, and a reduction in the relative standard deviation compared with formulations co-milled with either mannitol or cholesterol only, for remdesivir.

TABLE 11
Summary of the capsule characterization for Example 5.

| | Trial 1 | Trial 2 | Trial 3 | Trial 4 |
|---|---|---|---|---|
| Fill weight (mg) | 30.0 ± 1.5 | 33.3 ± 1.5 | 30.3 ± 1.5 | 33.7 ± 1.5 |
| Label claim (mg) | 30 | 30 | 30 | 30 |
| Device used | PowdAir | PowdAir | PowdAir | PowdAir |
| | (60 L/min) | (60 L/min) | (60 L/min) | (60 L/min) |
| ED (mg/capsule) | 3.6 ± 3.7 | 4.9 ± 5.4 | 17.2 ± 14.4 | 26.0 ± 1.7 |
| MMAD (μm) | 4.1 ± 0.4 | 3.8 ± 0.3 | 3.5 ± 0.1 | 3.7 ± 0.3 |
| GSD | 2.6 ± 0.5 | 2.6 ± 0.3 | 2.5 ± 0.6 | 2.2 ± 0.2 |

### Dissolution Data

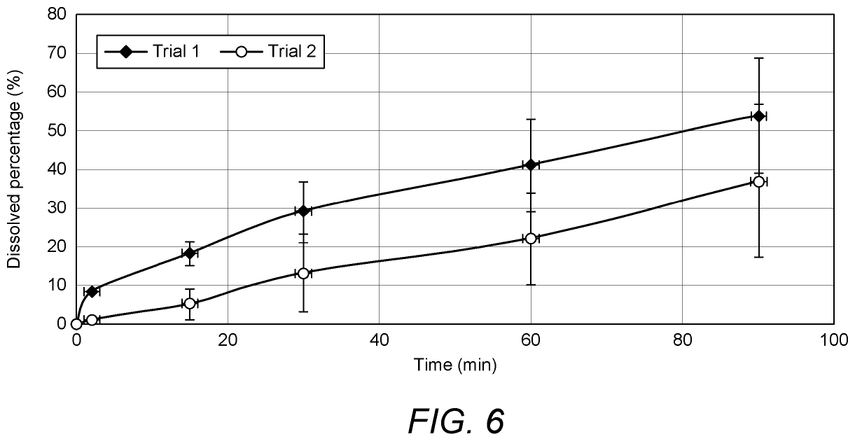

This is shown in FIG. 6. This shows dissolution profiles of Itraconazole formulations from Example 1 for Trial 1 (upper trace, containing excipients) and Trial 3 (lower trace, API only) performed in triplicate at the timepoints of 2, 15, 30, 60 and 90 minutes.

The dissolution media was a phosphate buffered saline (PBS) aqueous solution (Merck Millipore, Massachusetts, USA) at pH 7, maintained at 37° C. The saturation concentration of itraconazole in this media was 33 mg/L, therefore sink conditions were guaranteed below 11 mg/L. Dissolution was carried out in an apparatus II type dissolutor (Copley Scientific, Nottingham, UK) using a paddle-over-disk configuration, spinning at 75 rpm 3.5 cm above the disk for 2 h with 300 ml of dissolution media. Quantification was made by ultra-violet (UV) spectrophotometry in a Specord 200 Plus (Analytik Jena, Jena, Germany) at a wavelength of 265 nm in 0.5 s, using 1.5 ml quartz cells with a 10 mm light path. Other quantifications by UV were carried out using methanol as solvent.

### Methods and Further Definitions

### NGI Method

NGIs assessments were performed using 4 kPa pressure, 4 L volume and one actuation.

### Conditioning Step

In a typical conditioning step, formulations are stored under controlled conditions of temperature and relative humidity. Storage temperature is maintained above the glass transition temperature and relative humidity is set up to enable the water absorption by the dry powder and subsequent re-crystallization (see for example [19] Shetty et al, 2020).

### REFERENCES

[13] Liposomes and disaccharides as carriers in spray-dried powder formulations of superoxide dismutase, Lo Y; Tsai J, Kuo J
[2] X. M. Zeng, G. P. Martin, and C. Marriott. Particulate Interactions in Dry Powder Formulation for Inhalation. CRC Press, oct 2000. doi: 10.3109/9780203209592.
[1] J. Hu, Y. Dong, G. Pastorin, W. K. Ng, and R. B. Tan. Spherical agglomerates of pure drug nanoparticles for improved pulmonary delivery in dry powder inhalers. Journal of Nanoparticle Research, 15(4), 2013. ISSN 1572896X. doi: 10.1007/s11051-013-1560-2.
[4] M. L. H. Ong. The aerosol performance and physico-chemical properties of co-milled dry powder formulations for high dose delivery. PhD thesis, 2016. URL https://ses.library.usyd.edu.au/handle/2123/16277.
[3] J. Visser. Van der Waals and other cohesive forces affecting powder fluidization. Powder Technology, 58(1):1-10, 1989. ISSN 00325910. doi: 10.1016/0032-5910(89)80001-4.
[5] Z. Huang, L. Lin, C. Mc Goverin, H. Liu, L. Wang, Q. T. Zhou, M. Lu, and C. Wu. Dry powder inhaler formulations of poorly water-soluble itraconazole: A balance between in-vitro dissolution and in-vivo distribution is necessary. International Journal of Pharmaceutics, 551(1-2):103-110, 2018. ISSN 18733476. doi: 10.1016/j.ijpharm.2018.09.018. URL https://doi.org/10.1016/j.ijpharm.2018.09.018.
[7] G. Pilcer and K. Amighi. Formulation strategy and use of excipients in pulmonary drug delivery. International Journal of Pharmaceutics, 392(1-2):1-19, 2010. ISSN 03785173. doi: 10.1016/j.ijpharm.2010.03.017. URL http://dx.doi.org/10.1016/j.ijpharm.2010.03.017.
[9] S. Mangal, H. Park, R. Nour, N. Shetty, A. Cavallaro, D. Zemlyanov, K. Thalberg, V. Puri, M. Nicholas, A. S. Narang, and Q. T. Zhou. Correlations between surface composition and aerosolization of jet-milled dry powder inhaler formulations with pharmaceutical lubricants. International Journal of Pharmaceutics, 568, sep 2019. ISSN 18733476. doi: 10.1016/j.ijpharm.2019. 118504. URL https://pubmed.ncbi.nlm.nih.gov/31299339/.
[8] K. Stank and H. Steckel. Physico-chemical characterisation of surface modified particles for inhalation. International Journal of Pharmaceutics, 448(1):9-18, 2013. ISSN 03785173. doi: 10.1016/j.ijpharm.2013.03.009. URL http://dx.doi.org/10.1016/j.ijpharm.2013.03.009.
[10] M. George and I. Ghosh. Identifying the correlation between drug/stabilizer properties and critical quality attributes (CQAs) of nanosuspension formulation prepared by wet media milling technology. European Journal of Pharmaceutical Sciences, 48(1-2):142-152, 2013. ISSN 09280987. doi: 10.1016/j.ejps.2012.10.004. URL http://dx.doi.org/10.1016/j.ejps.2012.10.004.
[11] Y. Amharar, V. Curtin, K. H. Gallagher, E. O'Siochru, P. O'Connell, and A. M. Healy. Mitigating unwanted amorphisation: A screening method for the selection of suitable excipients. European Journal of Pharmaceutical Sciences, 81:181-188, 2016. ISSN 18790720. doi: 10.1016/j.ejps.2015. 10.016. URL http://dx.doi.org/10.1016/j.ejps.2015.10.016.
[6] S. Aziz, R. Scherlie, and H. Steckel. Development of high dose oseltamivir phosphate dry powder for inhalation therapy in viral pneumonia. Pharmaceutics, 12(12):1-21, 2020. ISSN 19994923. doi: 10.3390/pharmaceutics12121154.
[12] V. Curtin, Y. Amharar, Y. Hu, A. Erxleben, P. McArdle, V. Caron, L. Tajber, O. I. Corrigan, and A. M. Healy. Investigation of the capacity of low glass transition temperature excipients to minimize amorphization of sulfadimidine on comilling. Molecular Pharmaceutics, 10(1):386-396, jan 2013. ISSN 15438384. doi: 10.1021/mp300529a. URL https://pubs.acs.org/doi/abs/10.1021/mp300529a.
[14] X. Hu, C. Wang, L. Wang, Z. Liu, L. Wu, G. Zhang, L. Yu, X. Ren, P. York, L. Sun, J. Zhang, and H. Li. Nanoporous CD-MOF particles with uniform and inhalable size for pulmonary delivery of budesonide. International Journal of Pharmaceutics, 564(November 2018):153-161, 2019. ISSN 18733476. doi: 10.1016/j.ijpharm.2019.04.030.
[15] M. Malamatari, A. Charisi, S. Malamataris, K. Kachrimanis, I. Nikolakakis. Spray Drying for the Preparation of Nanoparticle-Based Drug Formulations as Dry Powders for Inhalation. MDPI Processes, 788 (2020): ISSN 2227-9717. doi: 10.3390/pr8070788
[16] J. Dolatabadi, H. Hamishehkar, H. Valizadeh. Development of dry powder inhaler formulation loaded with alendronate solid lipid nanoparticles: solid-state characterization and aerosol dispersion performance. Drug Development and Industrial Pharmacy, 41 (September 2014): 1431-1437. doi: 10.3109/03639045.2014.956111
[17] Adhikari et al, Solid state of inhalable high dose powders, Advanced Drug Delivery Reviews. 2022, 189:114468. doi:10.1016/j.addr.2022.114468.
[18] Lau et al, A review of co-milling techniques for the production of high dose dry powder inhaler formulation, Drug Development and Industrial Pharmacy. 2017. doi:10.1080/03639045.2017.1313858.
[19] Shetty et al, Physical stability of dry powder inhaler formulations, Expert Opin Drug Deliv. 2020, 17(1): 77-96. doi:10.1080/17425247.2020.1702643.
[20] Sibum et al, Challenges for pulmonary delivery of high powder doses, International Journal of Pharmaceutics. 2018, 548:325-336. Doi:10.1016/j.ijpharm.2018.07.008.

The invention claimed is:

1-38. (canceled)

39. A pharmaceutical composition comprising composite particles with a controlled aerodynamic particle size distribution, wherein the composite particles comprise one or more active pharmaceutical ingredients (API) in crystalline form, at least one sugar and at least one lipid;
wherein the composition is made by co-milling; and wherein the lipid comprises cholesterol;
and wherein the composition comprises the said sugar in an amount of from 0.5 to 45 wt % by weight of the total composition.

40. A pharmaceutical composition according to claim 39, wherein the particle size distribution of the composite particles is suitable for inhalation.

41. A pharmaceutical composition according to claim 39, wherein the particle size range is Dv90<20 μm.

42. A pharmaceutical composition according to claim 41, wherein the particle size range is Dv90<10 μm.

43. A pharmaceutical composition according to claim 39, wherein the particle size range is within 0.1 μm≤Dv90≤6 μm.

44. A pharmaceutical composition according to claim 39, wherein the sugar is chosen from the group comprising: mannitol, trehalose, trehalose hyclate, sucrose, lactose or raffinose, or a combination of two or more thereof.

45. A pharmaceutical composition according to claim 39, wherein the sugar is mannitol or trehalose, or a combination thereof.

46. A pharmaceutical composition according to claim 39, wherein the lipid further comprises a lipid chosen from the group comprising: saturated or unsaturated fatty acids; glycerides including neutral glycerides or phosphoglycerides; non-glyceride lipids such as steroids, waxes, or sphingolipids, or a combination of two or more thereof.

47. A pharmaceutical composition according to claim 39, wherein the lipid further comprises a lipid chosen from the group comprising a steroid selected from the following steroid classes: cholestanes, cholanes, pregnanes, androstanes, or estanes; or a phosphoglyceride chosen from the group comprising a phosphatidylcholine, a phosphatidylglycerol, or a phosphatidylethanolamine; or a combination of two or more thereof.

48. A pharmaceutical composition according to claim 39, wherein the lipid further comprises a phospholipid selected from dipalmitoylphosphatidylcholine (DPPC), distearoylphosphatidylcholine (DSPC), dimyristoylphosphatidylcholine (DMPC) or lecithin, or a combination of two or more thereof.

49. A pharmaceutical composition according to claim 39, wherein the sugar comprises mannitol or trehalose and the lipid comprises cholesterol.

50. A pharmaceutical composition according to claim 39, wherein the weight % of the components by weight of the total composition ranges as follows: API from 50 to 99.5 wt %; sugar from 0.5 to 45 wt %; and lipid from 0.01 to 5 wt %.

51. A pharmaceutical composition according to claim 39, wherein the weight % of the components by weight of the total composition ranges as follows: API from 80 to 99.5 wt %; sugar from 0.5 to 20 wt %; and lipid from 0.04 to 2 wt %.

52. A pharmaceutical composition according to claim 39, wherein the API is present at 30 wt % or more, based on the weight of the total composition.

53. A dry powder inhaler comprising a pharmaceutical composition according to claim 39.

54. A process for manufacturing a pharmaceutical composition according to claim 39, which process comprises the steps of:
a. Blending API and one or more excipients comprising at least one sugar or at least one lipid, or both at least one sugar and at least one lipid, into a homogeneous powder;
b. Reducing the particle size distribution of the blend;
wherein the composition is made by co-milling; and wherein the lipid comprises cholesterol.

55. A process according to claim 54, wherein step (b) is carried out without the use of a solvent.

56. A process according to claim 54, wherein step (b) comprises jet-milling.

57. A process according to claim 54, wherein the API and at least one sugar are first blended and jet-milled together, and at least one lipid is then blended and jet-milled with the resulting pharmaceutical composition, yielding a pharmaceutical composition comprising API, at least one sugar and at least one lipid.

58. A process according to claim 54, wherein no conditioning step is employed, or wherein a reduced amount of conditioning time is employed when compared with the conditioning time required to condition a composition comprising micronized API alone.
